(19) United States
(12) Patent Application Publication
(10) Pub. No.: US 2017/0027687 A1
(43) Pub. Date: Feb. 2, 2017

(54) EAR TIE

(71) Applicant: Northwood Medical Innovation Limited, London (GB)

(72) Inventors: Norbert Venantius KANG-BUDIALAM, London (GB); Marie-Claire HAINES, London (GB)

(21) Appl. No.: 15/303,193

(22) PCT Filed: Feb. 10, 2015
(86) PCT No.: PCT/GB2015/050357

§ 371 Date: Oct. 10, 2016

(30) Foreign Application Priority Data
Apr. 11, 2014 (GB) 1406554.4

(51) Int. Cl. A61F 2/18; A61F 11/00; A61F 2/00

(52) U.S. Cl. CPC A61F 2/18; A61F 2/0059; A61F 11/004; A61F 2002/183; A61F 2210/0014

(57) ABSTRACT

The present invention provides an ear tie (10) for insertion into a human ear to correct prominent ear deformity. The ear tie is formed from a shape-memory material and is pre-programmed in a first configuration to be in the form of a stilted arch comprising an arched portion (12) having a first and second end with a chord (14) therebetween, a stilt (16a, 16b) extending from the chord at each of the first and second ends of the arched portions and an engaging portion extending from the end of each stilt in the same plane thereof. In a second configuration, the ear tie is planar for insertion into an introducer. The introducer is inserted into the ear with the ear tie in the second configuration. The ear tie is then deployed from the introducer causing it to revert to the first, pre-programmed configuration.

### FIELD OF THE INVENTION

The present invention relates to an ear tie for reshaping the ear of a human.

### BACKGROUND OF THE INVENTION

Prominent ear deformity is common amongst the human population. An ear which projects more than 20 mm from the side of the head is increasingly perceived as prominent as this distance increases. By this estimate, up to 10% of the human population may be affected. It is desirable to correct the prominence very early in life when the cartilage is soft and pliable. However, often the prominence is ignored and not treated. Consequently, many children suffer the psychological consequences associated with prominent ears. These consequences can persist into adulthood.

The ear cartilage is cup-shaped and is formed of the conchal fossa and the antihelical fold which allows the ear to lie flat against the side of the head. Prominence may be the result of abnormal formation of the antihelical fold. Alternatively, it may be the result of a deep conchal fossa. One or both of the abnormalities may need to be addressed when correcting prominence of the ear. However, even for ears with a deep conchal bowl, accentuating the antihelical fold will make a difference to the perception of prominence. In most cases the prominence is apparent at birth.

International patent application number PCT/GB2012/000282 describes a scaffold formed of shape memory material in the shape of a horseshoe for reshaping the cartilaginous portion of an ear.

### SUMMARY OF THE INVENTION

The present invention provides an ear tie for reshaping the cartilaginous portion of an ear, the ear tie being formed from a shape memory material changeable between a first, pre-programmed configuration and a second configuration, wherein when in the first configuration the ear tie is in the shape of a stilted arch comprising an arched portion having a first and second end with a chord extending therebetween and a stilt extending from the chord at each of the first and second ends of the arch and wherein an engaging portion extends from an end of each stilt in the same plane thereof, and wherein when in the second configuration the ear tie is planar.

During surgery, precise control over deployment of the ear tie is critical to the resulting adjustment of prominence of an ear. If deployment were to occur inadvertently in the wrong position the patient's ear would not take the desired shape. The ear tie would then need to be removed and a further ear tie inserted. The configuration of the ear tie of the present invention enables each end of the ear tie to be deployed independently of the other end. This is advantageous as it enables a surgeon to make minor adjustments to the position of the ear tie once the first end is deployed thus limiting risk of the need to remove the ear tie due to deployment in the wrong position.

The engaging portion may comprise a pair of engaging members in the form of curved legs. The curved legs may be spatulate shaped with a broad and rounded end connected to an attenuated base.

The large surface area of the rounded end of each leg allows for adequate engagement of the ear tie with the cartilaginous portion of an ear. The attenuated portion of the leg provides flexibility to enable the legs to be compressed for insertion into an introducer.

The angle between the stilt and the chord may be 82.5 degrees. Such an angle has been found to apply sufficient pressure to the antihelical fold of an ear in order to re-shape the ear.

The ear tie may be gold plated to reduce visibility of the implant once deployed in the ear. The thickness of the gold plated layer may not exceed 4 microns.

The present invention also provides an introducer for inserting the ear tie into an ear comprising a catheter having a proximal end and a distal end and a lumen therethrough, wherein the lumen at the distal end of the catheter is sized to define a receiving formation for receiving an ear tie according to claims 1 to 19, and an applicator sized to insert into the lumen of the catheter and urge an ear tie to exit the catheter from the receiving formation thereof.

The introducer enables a surgeon to exert a high degree of control over how quickly and where the ear tie is deployed. This is advantageous as the risk of having to remove the ear tie in the event is deployed in the wrong position is reduced significantly.

The introducer may have indicator markings to identify when it is in the correct position to deploy the ear tie.

### BRIEF DESCRIPTION OF THE DRAWINGS

Specific embodiments of the present invention will now be described, by way of example only, with reference to the accompanying drawings in which:

FIG. 1a shows a front view of an ear tie in its first, pre-programmed configuration in the shape of a stilted arch;

FIG. 1b shows a plan view of the ear tie of FIG. 1a in its second configuration;

FIG. 1c shows an enlarged view of a portion of the end view of FIG. 1b showing detail of spatulate shaped engagement members;

FIG. 2 shows an illustrative view of an applicator for inserting the ear tie into a human ear;

FIGS. 3a and 3b show ear ties being positioned within a human ear.

### DETAILED DESCRIPTION OF THE INVENTION

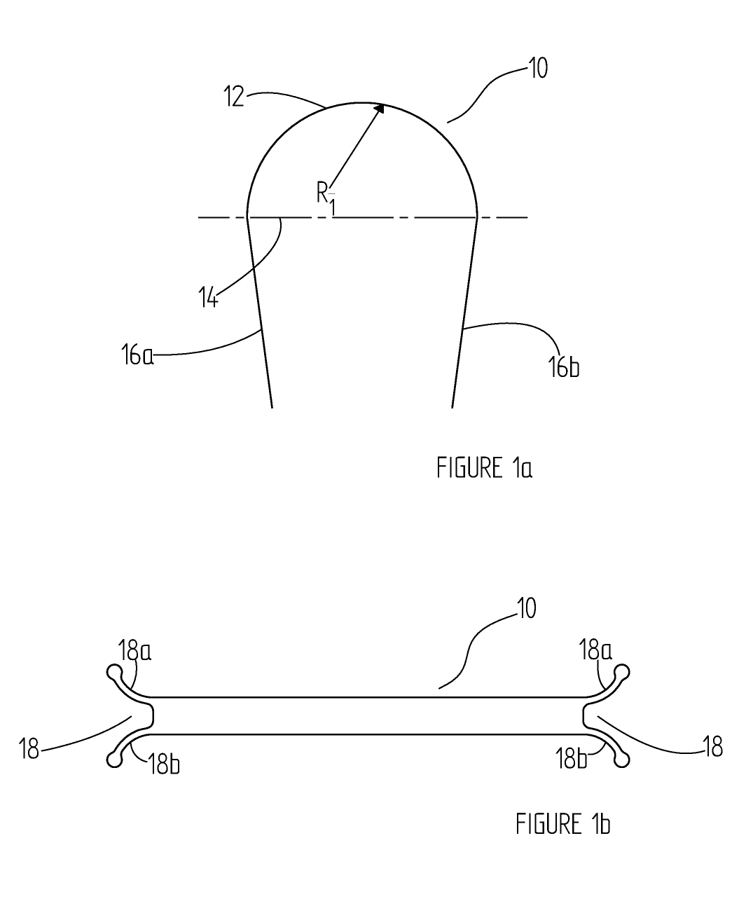

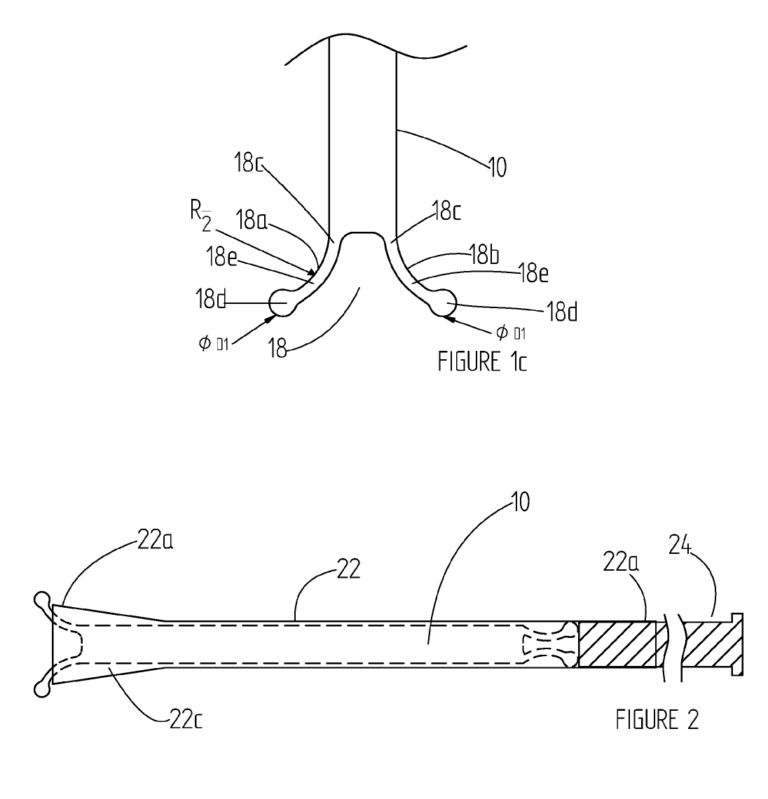

FIGS. 1a to 1c show an ear tie 10. The ear tie 10 is formed from a nickel titanium alloy, or other suitable shape memory material, and is pre-programmed into a first configuration, as shown in FIG. 1a, to take the shape of a stilted arch. The following description is by way of example only and is not intended to limit the scope of the invention. Values, where given, have been rounded up to one decimal place and are given in metric.

In the illustrated embodiment, the ear tie 10 is formed from a nickel titanium alloy of the type disclosed in U.S. Pat. No. 3,174,851, which is known as “Nitinol”. Details of such material can be found in NASA Publication SP 5110 entitled “55-NITINOL”—The Alloy with a Memory, Its Physical Metallurgy, Properties and Applications. C. M. Jackson et al, 1972. Many other materials having similar characteristics are well known to those skilled in the art.

The property of Nitinol which may be exploited in the present invention is the ability to pre-program a particular shape into the metal alloy and to activate the “memory” of this shape by heating/cooling it to specific temperatures. Using this property, it is possible to control the point at which the Nitinol changes shape.

The ear tie 10, when in the first configuration, comprises an arched portion 12 having a radius of curvature R₁ of between 2.4 and 2.6 millimetres. In the illustrated embodiment the radius of curvature R₁ of the arched portion is 2.52 millimetres. The arched portion 12 of the ear tie 10 takes the form of a semi-circle with the chord 14 of the semi-circle subtending both ends of the arched portion 12 of the ear tie 10.

A stilt 16a, 16b extends from each end of the arched portion 12 of the ear tie 10. In the illustrated embodiment, each stilt 16a, 16b is angled at 82.5° from the chord 14 of the semi-circle defined by the arched portion 12 of the ear tie 10. In other embodiments each stilt 16a, 16b may extend perpendicularly from said chord 14 or be positioned such that the angle between each stilt 16a, 16b and said chord 14 is between 0° and 180°. The stilts 16a, 16b of the ear tie 10 are of substantially equally length, width, thickness and form.

Referring to FIGS. 1b and 1c, the free end of each stilt 16a, 16b is provided with a portion 18 for engaging the cartilage of a patient's ear. This portion is in the form of a pair of curved legs 18a, 18b in the same plane as the respective stilt 16a, 16b. The curved legs 18a, 18b are spaced apart from one another and are symmetrical. Each leg 18a, 18b has a proximal end 18c which is attached to the end of a respective stilt 16a, 16b and a distal end 18d which is spatulate shaped. By spatulate shaped it is meant that the distal end 18d of each leg 18a, 18b is broad and rounded compared to the remainder of the leg 18a, 18b which is attenuated in comparison.

The radius of curvature R₂ of the each leg is between 1 and 1.4 millimetres. In the illustrated embodiment, the radius of curvature R₂ is 1.3 millimetres. The diameter D₁ of the rounded, distal end 18d of each leg is between 0.4 and 0.6 millimetres. In the illustrated embodiment the diameter D₁ is 0.5 millimetres. The width of the attenuated portion 18e of each leg 18a, 18b is between 0.1 and 0.4 millimetres. In the illustrated embodiment the width is 0.2 millimetres. The maximum distance between any part of the legs 18a, 18b is 3.5 millimetres. The longitudinal length of each leg 18a, 18b is between 3 and 3.3 millimetres. In the illustrated embodiment, the length is 3.2 millimetres.

The width of the arched portion 12 and stilts 16a, 16b of the stilted arch is between 1 and 1.4 millimetres. In the illustrated embodiment, the width is 1.3 millimetres. The thickness of the nitinol, or other, material that the ear tie 10 is formed from is approximately 0.3 millimetres thick.

The ear tie 10 is formed by laser cutting a flat sheet of nitinol, or other suitable material. The overall length of the laser cut ear tie is 17.5 to 18.5 millimetres thick. In the illustrated embodiment the length is 18 millimetres. The length of the laser cut ear tie excluding the legs defining the engaging portion 18 is between 14.6 and 15 mm. In the illustrated embodiment the length is 14.8 millimetres.

The laser cut ear tie is wrapped around tooling shaped to resemble the final ear tie 10 shape. Pressure is applied to each end of the laser cut ear tie to hold the ear tie in contact with the tooling. The laser cut ear tie and tooling is then heated to set the shape of the laser cut ear tie and form the stilted arch shape of the ear tie 10. When the ear tie 10 is cooled it retains the shape of the tooling it is wrapped around.

Once the ear tie 10 has cooled it is electropolished to achieve a wall thickness of approximately 0.2 millimetres.

The ear tie 10 is intended to be inserted into a patient's ear by way of an introducer 20. An example of a suitable introducer comprises a catheter 22 having a proximal end 22a and a distal end 22b with a lumen 22c therebetween. The lumen 22c is sized to be of greater diameter at the distal end 22b of the catheter 22 than at the proximal end 22a to define a receiving formation for receiving the ear tie 10. The catheter 22 may be straight or curved.

The lumen 22c of the catheter 22 is cooperable with an applicator 24 insertable therein. The applicator 24 comprises a rod, or plunger, which is slideable within the lumen 22c of the catheter 22 from the proximal end 22aa thereof towards the distal end 22b.

In use, the legs 18a, 18b defining the engaging portion 18 of one of the stilts 16a, 16b of the ear tie 10 are compressed together to allow the stilt 16a, 26b to be inserted into the distal end 22b of the catheter 22. The ear tie 10 is then almost fully inserted into the catheter 22 such that only the legs 18a, 18b defining the engaging portion 18 of the other stilt 16a, 16b, of the ear tie 10, remain exposed.

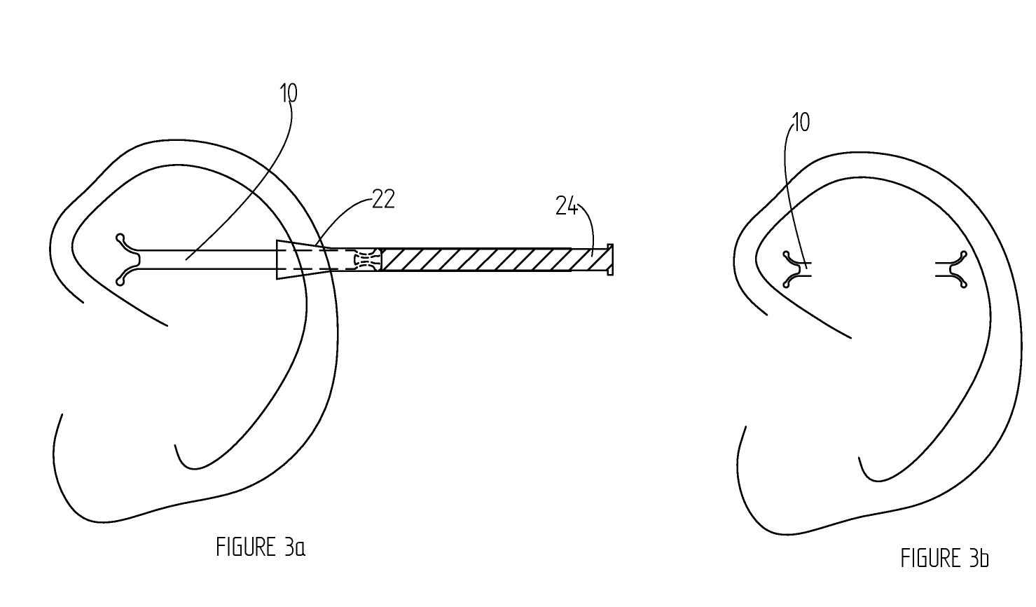

The ear tie 10 may be inserted into a human ear in accordance with the following steps:
a) inserting the ear tie 10 into the lumen 22c of the catheter 22;
b) sizing the ear to determine the desired position in the ear for the ear tie 10;
c) marking the intended position of the ear tie 10 on the ear;
d) making a first incision on the anterior surface of the ear;
e) inserting the catheter 22 into the incision and creating a tunnel through the cartilage of the ear and into the back of the ear;
f) making a subcutaneous tunnel behind the ear and perforating the cartilage of the antihelical fold of the ear;
g) creating a second incision in the antihelix adjacent to where the catheter 22 perforates the cartilage of the antihelical fold of the ear;
h) using the applicator 24, deploying the ear tie 10 (see FIGS. 3a and 3b);
i) removing the catheter 22 from the ear;
j) closing the first and second incisions.

The ear tie is deployed in two stages. Once the catheter has perforated the antihelical fold of the ear, the first end of the ear tie is deployed. As the catheter is deployed it reverts to the first, pre-programmed configuration. The surgeon can ensure that the first end of the ear tie is in the correct position before deploying the second end of the ear tie. As the ear tie 10 is deployed, the engaging portion 18 at the end of each stilt 16a, 16b engages with the cartilage of the ear either side of the antihelical fold to re-shape the ear.

1. An ear tie for reshaping the cartilaginous portion of an ear, the ear tie being formed from a shape memory material changeable between a first, pre-programmed configuration and a second configuration,
wherein when in the first configuration the ear tie is in the shape of a stilted arch comprising an arched portion having a first and second end with a chord extending therebetween and a stilt extending from the chord at each of the first and second ends of the arch and wherein an engaging portion extends from an end of each stilt in the same plane thereof,
and wherein when in the second configuration the ear tie is planar.

2. An ear tie for reshaping the cartilaginous portion of an ear according to claim 1, wherein the engaging portion comprises a pair of engaging members.

3. An ear tie for reshaping the cartilaginous portion of an ear according to claim 2, wherein the pair of engaging members comprises two legs spaced apart from one another, each leg having a proximal end attached to a stilt of the stilted arch and a distal end.

4. An ear tie for reshaping the cartilaginous portion of an ear according to claim 3, wherein each leg is curved in the same plane as the stilt of the stilted arch.

5. An ear tie for reshaping the cartilaginous portion of an ear according to claim 4, wherein the distal end of each leg is spatulate shaped.

6. An ear tie for reshaping the cartilaginous portion of an ear according to claim 5, wherein each stilt of the stilted arch is configured such that it is at an acute angle to the chord of the arched portion of the stilted arch.

7. An ear tie for reshaping the cartilaginous portion of an ear according to claim 6, wherein the angle between the stilt and the chord is 7.5 degrees.

8. An ear tie for reshaping the cartilaginous portion of an ear according to claim 7, wherein the stilt extending from the chord at the first end of the arched portion is substantially identical to the stilt extending from the chord at the second end of the arched portion.

9. An ear tie for reshaping the cartilaginous portion of an ear according to claim 8, wherein the radius of curvature of the arched portion is between 2.4 and 2.6 millimetres.

10. An ear tie for reshaping the cartilaginous portion of an ear according to claim 9 formed from nitinol.

11. An ear tie for reshaping the cartilaginous portion of an ear according to claim 10 having a thickness of between 0.2 and 0.3 millimetres.

12. An ear tie for reshaping the cartilaginous portion of an ear according to claim 11 wherein the width of the arched portion and the stilts is between 1 and 1.4 millimetres.

13. An ear tie for reshaping the cartilaginous portion of an ear according to claim 4, wherein the maximum width between the distal end of each leg is 3.6 millimetres.

14. An ear tie for reshaping the cartilaginous portion of an ear according to claim 13, wherein the radius of curvature of each leg is between 1 and 1.4 millimetres.

15. An ear tie for reshaping the cartilaginous portion of an ear according to claim 5, wherein the spatulate shaped end of each leg comprises a broad, rounded end and a narrow, attenuated base.

16. An ear tie for reshaping the cartilaginous portion of an ear according to claim 15, wherein the broad rounded end of the spatulate shaped end of each leg has a diameter of 0.4 to 0.6 millimetres.

17. An ear tie for reshaping the cartilaginous portion of an ear according to claim 14, wherein the attenuated base of the spatulate shaped end of each leg has a width of 0.15 to 0.3 millimetres.

18. An ear tie for reshaping the cartilaginous portion of an ear according to claim 12 wherein the ear tie is electropolished.

19. An ear tie for reshaping the cartilaginous portion of an ear as described with reference to, and/or as shown in, the drawings.

20. An introducer for inserting an ear tie into an ear comprising:
a catheter having a proximal end and a distal end and a lumen therethrough, wherein the lumen at the distal end of the catheter is sized to define a receiving formation for receiving an ear tie according to claims 1, and
an applicator sized to insert into the lumen of the catheter and urge an ear tie to exit the catheter from the receiving formation thereof.

21. An introducer according to claim 20 further comprising a visual indicator to identify how far into a patient's ear the introducer has been inserted.

22. An introducer as described with reference to, and/or as shown in, the drawings.
